(19) United States
(12) Patent Application Publication
(10) Pub. No.: US 2017/0239167 A1
(43) Pub. Date: Aug. 24, 2017

(54) Hair Products Containing Polyetheramine

(71) Applicant: Colgate-Palmolive Company, New York, NY (US)

(72) Inventors: Ramon Arnoldo Lopez Baca, Del.Miguel Hidalgo, DF (MX); Yumiko Raquel GARCIA MACIAS, Del.Miguel Hidalgo, DF (MX)

(73) Assignee: Colgate-Palmolive Company, New York, NY (US)

(21) Appl. No.: 15/504,669

(22) PCT Filed: Aug. 18, 2014
(86) PCT No.: PCT/US2014/051464

§ 371 Date: Feb. 17, 2017

(51) Int. Cl. A61K 8/86; A61Q 5/12; A61K 8/34

(52) U.S. Cl. CPC A61K 8/86; A61K 8/342; A61Q 5/12

(57) ABSTRACT

The present disclosure pertains to compositions that include a linear polyether polymer, methods involving the application of such compounds to hair, methods for making hair conditioners using such compounds, and uses of compositions containing such compounds for conditioning of hair for increasing softness. The linear polyether polymer has a weight average less than 5000 and terminates with —N—(—CH2-CH(OH)—CH2-CI)2.

### BACKGROUND

There presently exist a wide variety of consumer products for cleansing and conditioning of hair. Hair conditioners include leave-on and rinse-off products. Shampoos may consist of dedicated products that are solely for cleansing, or may include hair conditioning agents that permit a single product to both cleanse and condition the hair. Other products that are not intended solely as cleansing or conditioning compositions may include conditioning agents. The active components of conditioning products may be included to enhance the softness, manageability, luster, or moisture content of hair, to reduce dryness, tangling, brittleness, or color loss, or to provide any combination of these benefits. While hair conditioners initially consisted of naturally-occurring oils, modern hair care products can make use of silicone, fatty alcohols, quaternary ammonium compounds, and other actives in order to accomplish a conditioning effect.

Many existing conditioning products may be effective to provide one or several particular benefits, but are unable to confer another desirable effect. Accordingly, there exists an ongoing need for conditioning agents and products that are made from such agents that provide any of the traditionally desired benefits of hair conditioning.

### BRIEF SUMMARY

The present disclosure concerns methods comprising applying to the hair of a mammal a composition that includes a linear polyether polymer having a weight average molecular weight less than 5000 that terminates with —N—(CH₂—CH(OH)—CH₂—Cl)₂.

Also disclosed are compositions for the conditioning of hair, methods for making a hair conditioner, and uses of a composition for conditioning of hair, each of which involve a linear polyether polymer of the above-described variety.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Provided herein are methods comprising applying to the hair of a mammal a composition that includes a linear polyether polymer having a weight average molecular weight less than 5000 that terminates with —N—(—CH₂—CH(OH)—CH₂—Cl)₂.

Passerelle™ DFD available from Devan Chemicals (France), and represents an exemplary composition that includes a linear polyether polymer according to the present disclosure. Passerelle™ DFD contains 82% of DP5270 (also produced by Devan Chemicals) and ethoxylated fatty-quaternary softeners. The DP5720 product is sold as an aqueous composition that contains 20% linear polyether polymer with a total solids complement of 23-24%, with the other solids being surfactants.

The linear polyether polymer of the present methods may have a weight average molecular weight less than 5000, less than 4500, less than 4000, less than 3500, less than 3000, less than 2500, less than 2000, less than 1500, or less than 1000.

The linear polyether polymer may be present in an amount of 0,01% to 10.0% by weight, 0.05% to 5.0% by weight, 0.10% to 3.0% by weight, 0.25% to 2.0% by weight, 0.3 to 1.5% by weight, or 0.35% to 0.5% by weight of the composition of the presently disclosed methods. For example, the linear polyether polymer may be present in an amount of 0.35% by weight of the composition.

The compositions of the present methods may further comprise one or more of a thickener, a viscosity enhancer, a pH modifier, a surfactant, an emulsifier, an oil, a detangling agent, a moisturizer, a hair shine enhancer, a hair strength enhancer, a preservative, and fragrance. For example, the compositions may comprise each of a thickener, a viscosity enhancer, a pH modifier, a surfactant, an emulsifier, an oil, a detangling agent, a moisturizer, a hair shine enhancer, a hair strength enhancer, a preservative, and fragrance. In a certain embodiment, the composition comprises water, hydrophobically modified hydroxyethylcellulose, cetyltrimethyammonium chloride, cetyl stearyl alcohol, glyceryl monostearate, mineral oil, a cationic silicone emulsion, honey, hydrolyzed silk, a preservative, fragrance, and NaOH and citric acid. The compositions may take the form of a hair conditioner, such as a rinse-off hair conditioner.

The compositions of the present methods may comprise fatty alcohols, such as stearyl alcohol, cetyl alcohol, or both. The compositions may also or alternatively include silicones, such as dimethicone, dimethiconol, cyclopentasiloxane, or any combination thereof. The compositions may also or alternatively include cationics, including surfactants like cetrimonium chloride or quaternium 82, or polymeric cationics, such as the polyquaternium series (e.g., polyquaternium-6, -7, or -8) or cationic guar gum. Further examples of fatty alcohols, silicones, and cationics are well known among those skilled in formulating hair products.

The present disclosure also pertains to compositions for the conditioning of hair comprising a linear polyether polymer having a weight average molecular weight less than 5000 that terminates with —N—(—CH₂—CH(OH)—CH₂—Cl)₂, and, one or more further ingredients that are suitable for inclusion in said composition for use on the hair of a mammal.

Unless otherwise specified, any component, element, attribute, or step that is disclosed with respect to one embodiment of the present methods, compositions, and uses may apply to any other embodiment that is disclosed herein. For example, the presently disclosed compositions may have any of the characteristics of the composition that are used pursuant to the present methods, and vice versa, and the compounds that are used in the presently disclosed methods may have any of the characteristics of the compounds of the presently disclosed compositions, and vice versa.

Thus, the linear polyether polymer in the present compositions may have any of the characteristics of the linear polyether polymers described above with respect to the disclosed methods that involve the use of a linear polyether polymer. In addition, the present compositions may have any of the characteristics of the compositions that are used pursuant to the above-described methods.

As used herein, one or more further ingredients that are suitable for inclusion in the composition for use on the hair of a mammal refers to any ingredient that is characteristic of a hair product, such as a hair conditioner. The present compositions may also include components that are not necessarily characteristic of a hair product, but must include at least one component that is characteristic of a hair product. Exemplary ingredients that are characteristic of a hair product include fatty alcohols, silicones, and cationics. Examples of these ingredients are described above in connection with the present methods, and further examples of which are well known among those skilled in formulating hair products. The component that is characteristic of a hair product may be, but is not necessarily, an ingredient that is not typically included in a non-hair product, such as in a fabric conditioner.

The present disclosure also pertains to methods for making a hair conditioner comprising preparing a composition that includes a linear polyether polymer, as previously described, and one or more further ingredients that are suitable for inclusion in the composition for use on the hair of a mammal. The linear polyether polymer in the present methods may have any of the characteristics of the compounds described above with respect to the above described methods that involve the use of a linear polyether polymer. In addition, the compositions of the present methods may have any of the characteristics of the compositions that are used pursuant to the above-described methods. Likewise, the one or more further ingredients that are suitable for inclusion in the composition for use on the hair of a mammal may have any of the characteristics that are described in connection with the above-described compositions, such as in the immediately preceding paragraph.

Also disclosed herein are uses of a composition for conditioning of hair for increasing the softness of the hair, wherein the composition comprises a linear polyether polymer, as previously described. For example, the composition of the presently disclosed uses may be a hair conditioner, such as a rinse-off hair conditioner. The composition may further include one or more further ingredients that are suitable for inclusion in the composition for use on the hair of a mammal. The linear polyether polymer in the present uses may have any of the characteristics of the compounds described above with respect to the above described methods that involve the use of a linear polyether polymer. In addition, the compositions of the present uses may have any of the characteristics of the compositions that are used pursuant to the above-described methods. Likewise, the one or more further ingredients that are suitable for inclusion in the composition for use on the hair of a mammal may have any of the characteristics that are described in connection with the above-described compositions.

### EXAMPLES

### Example 1

Clean, bleached hair was prepared into 3.5±0.1 g tresses and pretreated with a cleansing solution of 20% ammonium lauryl sulfate, and then dried overnight.

Hair tresses were wet under tap water at 25° C. and then treated with 0.5 mL of a rinse-off conditioner prototype (see Table 1, below) containing either a linear polyether polymer having a weight average molecular weight less than 5000 that terminates with —N—(—CH₂—CH(OH)—CH₂—Cl)₂, or a comparison active, each at 0.35% by weight of the hair conditioner. Hair was rubbed for one minute with the test conditioner, and then rinsed again under tap water for one further minute.

The rinse-off conditioner formula is listed in Table 1 below, and included 0.35% by weight of Silsoft HC400 amino bispropyl dimethicone (used for comparative purposes) or 0.35% by weight of a linear polyether polymer having a weight average molecular weight less than 5000 that terminates with —N—(—CH₂—CH(OH)—CH₂—Cl)₂. Passerelle™ DFD (Devon Chemicals, France) was used as the source of the linear polyether polymer.

Table 1, below, lists the components of the tested conditioner formulas, based on active amount.

TABLE 1

| Component | Weight % | Weight % |
|---|---|---|
| Water and minors | Q.S. | Q.S. |
| (fragrance, pH agent, preservative, etc.) | | |
| Stearyl Alcohol | 3.15 | 3.15 |
| Cetyl Alcohol | 1.35 | 1.35 |
| Cetrimonium Chloride | 1 | 1 |
| Mineral Oil | 0.5 | 0.5 |
| Glyceryl Stearate | 0.45 | 0.45 |
| Silsoft HC400 Amino bispropyl dimethicone | 0.35 | 0 |
| or | | |
| Linear polyether polymer | 0 | 0.35 |

Seven trained panelists evaluated three tresses and organized the tresses in terms of hair softness, from lowest to highest degree of softness. The panelists assigned a grade to each tress that was based on the degree of softness: a grade of 3 was assigned to the softest tress, a grade of 2 was assigned to the next softest tress, and a grade of 1 was assigned to the tress that was the least soft of the three test samples. The grades for each treatment type were combined and the respective grades for the treatment types were compared with one another. Table 2 shows the results of the comparison of the softness resulting from treatment using the various test compositions.

TABLE 2

| Composition | Hair Softness |
|---|---|
| Comparative | 16 |
| Inventive | 20 |

As can he seen in Table 2, the inventive composition with the linear polyether polymer having a weight average molecular weight less than 5000 that terminates with —N—(—CH₂—CH(OH)—CH₂—Cl)₂ provided a higher degree of softness as compared to the comparative composition.

What is claimed is:

1. A method comprising:
applying to hair of a mammal a composition comprising a linear polyether polymer having a weight average molecular weight less than 5000 that terminates with —N—(—CH₂—CH(OH)—CH₂—Cl)₂.

2. The method of claim 1, wherein the linear polyether polymer has a weight average molecular weight of less than 4000.

3. The method of claim 1, wherein the linear polyether polymer has a weight average molecular weight of less than 3000.

4. The method of claim 1, wherein the linear polyether polymer has a weight average molecular weight of less than 2000,

5. The method of claim 1, wherein the composition comprises 0.01% to 10.0% by weight of the linear polyether polymer.

6. The method of claim 1, wherein the composition comprises 0.3 to 0.4% by weight of the linear polyether polymer.

7. The method of claim 1, wherein the composition comprises about 0.35% by weight of the linear polyether polymer.

8. The method of claim 1, wherein the composition further comprises a fatty alcohol.

9. The method of claim 8, wherein the fatty alcohol comprises stearyl alcohol, cetyl alcohol, or a mixture of stearyl alcohol and cetyl alcohol.

10. A composition for conditioning mohair comprising:
a linear polyether polymer having a weight average molecular weight less than 5000 that terminates with —N—(—CH₂—CH(OH)—CH₂—Cl)₂; and
one or more further ingredients that are suitable for inclusion in the composition for use on the hair of a mammal.

11. The composition of claim 10 wherein the linear polyether polymer has a weight average molecular weight of less than 4000.

12. The composition according of claim 10, wherein the linear polyether polymer has a weight average molecular weight of less than 3000.

13. The composition of claim 10, wherein the linear polyether polymer has a weight average molecular weight less than 2000.

14. The composition of claim 10, wherein the composition comprises 0.01% to 10.0% by weight of the linear polyether polymer.

15. The composition of claim 10, comprising 0.3 to 0.4% by weight of the linear polyether polymer.

16. The composition of claim 10, comprising about 0.35% by weight of the linear polyether polymer.

17. The composition of claim 10, wherein the further ingredient comprises a fatty alcohol.

18. The composition of claim 17, wherein the fatty alcohol comprises stearyl alcohol, cetyl alcohol, or a mixture of stearyl alcohol and cetyl alcohol.

19. The composition of claim 10, wherein the composition is a rinse-off hair conditioner.

20. A method for making the composition of claim 10, comprising mixing
the linear polyether polymer and
the one or more further ingredients.

21. (canceled)
